(19) United States
(12) Patent Application Publication
(10) Pub. No.: US 2019/0224423 A1
(43) Pub. Date: Jul. 25, 2019

(54) SYRINGE WITH ADJUSTABLE NEEDLE SHIELD

(71) Applicant: KING SAUD UNIVERSITY, Riyadh (SA)

(72) Inventor: MASHAEL ABDULAZIZ ALHADLAQ, RIYADH (SA)

(21) Appl. No.: 15/878,371

(22) Filed: Jan. 23, 2018

(51) Int. Cl. A61M 5/32; A61M 5/315; A61M 5/31; A61M 5/24

(52) U.S. Cl. CPC A61M 5/3245; A61M 5/315; A61M 2005/247; A61M 5/2466; A61M 5/3129

(57) ABSTRACT

The syringe with an adjustable needle shield is a reusable syringe, such as for applying anesthetic to a patient during a dental procedure, with a sliding needle shield, allowing the exposed length, or application length, of the needle to be selectively adjusted. The syringe with an adjustable needle shield includes an elongated hollow barrel having opposed open and closed ends, a medicament cartridge removably received within the elongated hollow barrel, and a hypodermic needle. A first end of a plunger rod is slidably received within the elongated hollow barrel and is adapted for selectively compressing a sliding plunger end of the medicament cartridge. A cylindrical shield is provided for selectively and adjustably covering the hypodermic needle. The elongated hollow barrel slidably engages the cylindrical shield such that the cylindrical shield adjustably covers a desired portion of the hypodermic needle.

### BACKGROUND

### 1. Field

The disclosure of the present patent application relates to medical syringes, and particularly to a syringe with a sliding needle shield, allowing an exposed length of the needle to be selectively adjusted.

### 2. Description of the Related Art

FIG. 2 illustrates a conventional prior art reusable syringe 100, such as those typically used in dentistry for the application of a local anesthetic. The reusable syringe 100 is a breech-loading metal cartridge syringe, in which a hermetically sealed glass cartridge 102 containing the anesthetic solution fits into the breech 104 of barrel 110 of syringe 100. A disposable needle 106 is screwed into the front of the syringe 100. The length of the needle 106, which extends into the breech 104, penetrates a rubber membrane or metal cap on the front of cartridge 102 and extends into the anesthetic solution contained in glass cartridge 102. A plunger rod 108 is then forced into the breech 104 of the syringe 100 under pressure generated by the user's thumb, for example, engaging thumb ring 112. As is well known, the plunger rod 108 is tipped with an actuating element 114 (typically referred to as a “harpoon”), which is adapted for pushing a plunger end of the cartridge 102. Syringe cartridges 102 are typically manufactured with a sliding rubber plug in the plunger end (opposite the end pierced by needle 106). The actuating element 114, or “harpoon”, pushes the sliding rubber plug within cartridge 102, forcing the liquid contents of cartridge 102 to be expelled through the needle 106.

As with most typical syringes, the application length of needle 106 remains fixed. However, syringes, such as reusable syringe 100, are rarely used for only a singular purpose; i.e., syringes are typically used for a wide variety of medical procedures on a wide variety of differing body parts. A desired length of the needle is dependent upon the particular procedure and/or body part. As such, multiple needles are often used with a single syringe. The removal and replacement of a needle can be time-consuming and often results in needle stick injuries. Thus, a syringe with an adjustable needle shield solving the aforementioned problems is desired.

### SUMMARY

The syringe with an adjustable needle shield is a reusable syringe, such as those typically used for applying anesthetic to a patient during a dental procedure, with a sliding needle shield, allowing the exposed length, or application length, of the needle to be selectively adjusted. The syringe with an adjustable needle shield includes an elongated hollow barrel having opposed open and closed ends, a medicament cartridge removably received within the elongated hollow barrel, and a hypodermic needle. The hypodermic needle is secured to, and extends through, the closed end of the elongated hollow barrel. A first end of the hypodermic needle is free, and an opposed second end of the hypodermic needle is positioned within the elongated hollow barrel for piercing a seal end of the medicament cartridge.

A first end of a plunger rod is slidably received within the elongated hollow barrel and is adapted for selectively compressing a sliding plunger end of the medicament cartridge. The plunger rod at least partially projects through the open end of the elongated hollow barrel, with the second end of the plunger rod being adapted for manual pushing by a user's finger. A cylindrical shield is provided for selectively and adjustably covering the hypodermic needle. The cylindrical shield is hollow and has opposed first and second open ends. The closed end of the elongated hollow barrel is slidably received within the cylindrical shield through the second open end such that the cylindrical shield adjustably covers a desired portion of the hypodermic needle, and the first end remains free.

A hollow cylindrical mount, having opposed first and second open ends, is further provided, with the first open end being free and the second open end being secured to the closed end of the elongated hollow barrel. The hypodermic needle partially extends through the hollow cylindrical mount. An engaging member is secured to an exterior face of the hollow cylindrical mount by an elastic member. The engaging member is selectively and releasably received through a selected one of a plurality of openings formed through the cylindrical shield to releasably lock an axial position of the cylindrical shield with respect to the elongated hollow barrel. The releasable and adjustable locking of the cylindrical shield with respect to the elongated hollow barrel allows the user to selectively adjust the exposed length, or application length, of the hypodermic needle.

These and other features of the present invention will become readily apparent upon further review of the following specification.

### BRIEF DESCRIPTION OF THE DRAWINGS

FIG. 1 is a side view of a syringe with an adjustable needle shield.

FIG. 2 is a side view in partial section of a typical prior art reusable syringe.

FIG. 3A is a partial side view in section of the syringe with an adjustable needle shield, shown in a fully covered configuration.

FIG. 3B is a partial side view in section of the syringe with an adjustable needle shield, with the needle shield shown in a partially retracted configuration.

FIG. 3C is a partial side view in section of the syringe with an adjustable needle shield, with the needle shield shown in a fully retracted configuration.

Similar reference characters denote corresponding features consistently throughout the attached drawings.

### DETAILED DESCRIPTION OF THE PREFERRED EMBODIMENTS

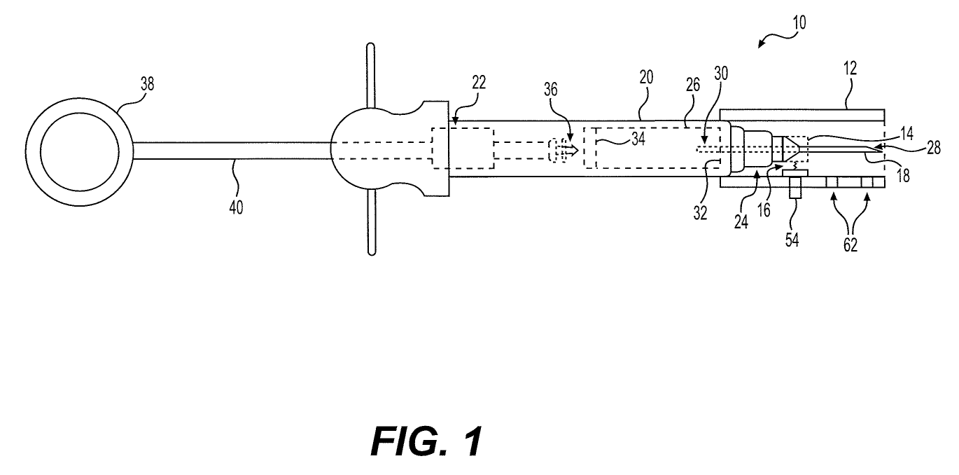

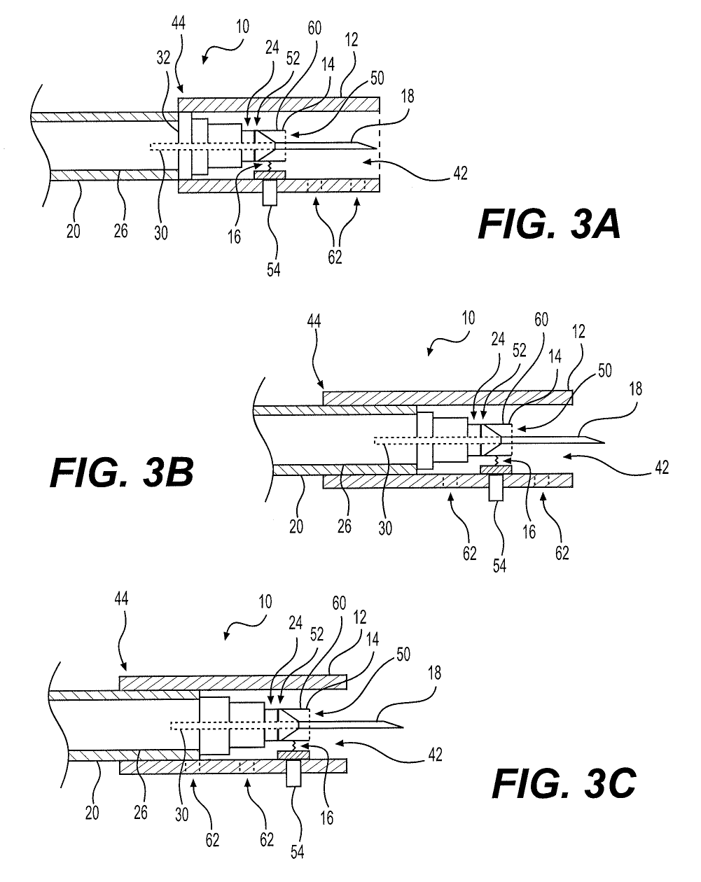

Referring now to FIGS. 1 and 3A-3C, there is shown a syringe with an adjustable needle shield 10. The syringe with an adjustable needle shield 10 can be configured for one or more medical uses, such as for applying anesthetic to a patient during a dental procedure. As will be described in greater detail below, the syringe with an adjustable needle shield 10 includes a needle shield 12 which allows the exposed length, or application length, of the hypodermic needle 18 to be selectively adjusted. It should be understood that the overall contouring, configuration and relative dimensions of the syringe 10 are shown in FIGS. 1 and 3A-3C for exemplary purposes only, and that needle shield 12, and its associated locking elements (to be described in greater detail below), may be applied to any suitable type of syringe.

Similar to a conventional reusable, cartridge-type syringe, the syringe with an adjustable needle shield 10 includes an elongated hollow barrel 20 having opposed open and closed ends 22, 24, respectively, a medicament cartridge 26 removably received within the elongated hollow barrel, and a hypodermic needle 18. It should be understood that medicament cartridge 26 and hypodermic needle 18 may be any suitable type of medicament cartridge and hypodermic needle, as is well known in the art. The hypodermic needle 18 is secured to, and extends through, the closed end 24 of the elongated hollow barrel 20 in a conventional manner. A first end 28 of hypodermic needle 18 is free and is adapted for piercing the skin of the patient. An opposed second end 30 of hypodermic needle 18 is positioned within the elongated hollow barrel 20 for piercing a seal end 32 of the medicament cartridge 26, as is conventionally known.

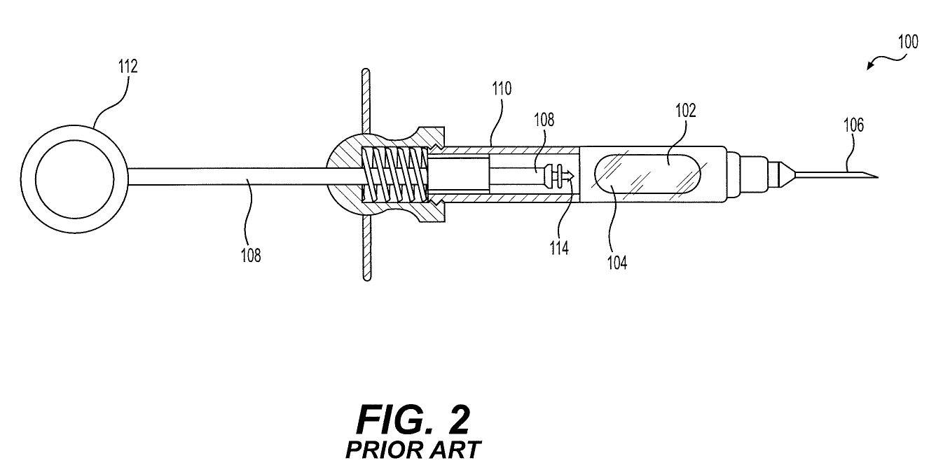

A first end 36 of a plunger rod 40 is slidably received within the elongated hollow barrel 20 and is adapted for selectively compressing a sliding plunger end 34 of the medicament cartridge 26 in a manner similar to that of conventional reusable, cartridge-type syringes. Although first end 36 is shown configured as a conventional harpoon, similar to that of FIG. 2, it should be understood that the harpoon end is shown for exemplary purposes only, and that first end 36 may be configured in any suitable manner. The plunger rod 40 at least partially projects through the open end 22 of the elongated hollow barrel 20, with the second end 38 of the plunger rod 40 being adapted for manual pushing by a user's finger. Although shown in FIG. 1 as having a conventional thumb ring, it should be understood that second end 38 of plunger rod 40 may be configured in any desired manner to allow the user to easily manipulate the sliding of plunger rod 40 within the elongated hollow barrel 20.

As best shown in FIGS. 3A-3C, cylindrical shield 12 is provided for selectively and adjustably covering the hypodermic needle 18. The cylindrical shield 12 is hollow and has opposed first and second open ends 42, 44. The closed end 24 of the elongated hollow barrel 20 is slidably received within the cylindrical shield 12 through the second open end 44 such that the cylindrical shield 12 adjustably covers a desired portion of the hypodermic needle 18, and the first end 42 of cylindrical shield 12 remains free. The cylindrical shield 12 is selectively and releasably lockable with respect to the elongated hollow barrel 20. In an embodiment, a plurality of openings 62 extend through the cylindrical shield 12 for selectively receiving an engaging member 54, as described below in detail.

A hollow cylindrical mount 14, having opposed first and second open ends 50, 52, respectively, is further provided. The first open end 50 of the hollow cylindrical mount 14 is free and the second open end 52 is secured to the closed end 24 of the elongated hollow barrel 20. As shown, the hypodermic needle 18 partially extends through the hollow cylindrical mount 14, with hollow cylindrical mount 14 and cylindrical shield 12 being concentric with respect to one another, and with hypodermic needle 18 extending along the axial center of each of hollow cylindrical mount 14 and cylindrical shield 12.

An engaging member 54 is secured to an exterior face 60 of the hollow cylindrical mount 14 by an elastic member 16. Although elastic member 16 is shown as a spring, it should be understood that engaging member 54 may be secured to exterior face 60 by any suitable type of elastic tensioning element. The engaging member 54 is selectively and releasably received through a selected one of the plurality of openings 62 to releasably lock an axial position of the cylindrical shield 12 with respect to the elongated hollow barrel 20. Once secured within a selected opening 62, the engaging member 54 can be depressed to permit sliding of the cylindrical shield 12 and insertion of the engaging member 54 into a different opening 62, if desired. It should be understood that a length of the engaging member 54 can be significantly smaller than that depicted in the drawings, to facilitate disengagement and movement of the cylindrical shield 12. Preferably, as shown, the engaging member 54 has a substantially T-shaped cross section. Although only three axially-aligned openings 62 are shown, there may be any suitable number of openings 62 arrayed in any suitable configuration.

The releasable and adjustable locking of the cylindrical shield 12 with respect to the elongated hollow barrel 20 allows the user to selectively adjust the exposed length, or application length, of the hypodermic needle 18. In the configuration of FIG. 3A, the cylindrical shield 12 is fully extended, with engaging member 54 engaging the left-most opening 62 (in the orientation of FIG. 3A). This configuration provides complete coverage of hypodermic needle 18, preventing accidental injury to the user. In the configuration of FIG. 3B, hypodermic needle 18 is partially exposed, allowing the hypodermic needle 18 to be used when an exposure length, or application length, of needle 18 is required to be relatively small. In this configuration, the engaging member 54 engages the center opening 62 (in the three-opening example of FIGS. 3A-3C). In the configuration of FIG. 3C, the cylindrical shield 12 is fully retracted, with engaging member 54 engaging the right-most opening 62 (in the orientation of FIG. 3C). This configuration provides maximum exposure length for hypodermic needle 18.

It is to be understood that the syringe with an adjustable needle shield is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1-5. (canceled)

6. A syringe with an adjustable needle shield, comprising:
an elongated hollow barrel having opposed open and closed ends, the elongated hollow barrel having a longitudinal axis;
a medicament cartridge removably received within the elongated hollow barrel;
a hypodermic needle extending through the closed end of the elongated hollow barrel, the hypodermic needle having opposed first and seconds ends, the first end thereof being free and the second end thereof being positioned within the elongated hollow barrel and being adapted for piercing a seal end of the medicament cartridge;
a plunger rod having opposed first and second ends, the first end thereof being slidably received within the elongated hollow barrel and being adapted for selectively compressing a sliding plunger end of the medicament cartridge, the plunger rod at least partially projecting through the open end of the elongated hollow barrel, the second end of the plunger rod being adapted for manual pushing by a user's finger;
the adjustable needle shield consisting of a cylindrical tubular body having opposed first and second open ends, wherein the cylindrical tubular body has an outer peripheral surface extending continuously from the first to the second open ends, the closed end of the elongated hollow barrel being slidably received within the shield through the second open end such that the shield adjustably covers a desired portion of the hypodermic needle and the first open end being free, the shield having three axially aligned locking openings formed through the outer peripheral surface;
wherein the three axially aligned locking openings comprises a proximal locking opening, a distal locking opening, and an intermediate locking opening between the proximal locking opening and the distal locking opening;
further wherein the cylindrical tubular body has a first inner diameter between the second end and the proximal locking opening and a second inner diameter between the proximal locking opening and the intermediate locking opening, wherein the first inner diameter and the second inner diameter are the same;
a hollow cylindrical mount having opposed first and second open ends, the first open end being free and the second open end being secured to the closed end of the elongated hollow barrel, the hypodermic needle partially extending through the hollow cylindrical mount;
a single engaging member; and
a single elastic element securing the single engaging member to an exterior face of the hollow cylindrical mount, wherein the engaging member is selectively and releasably received through a selected one of the three locking openings formed through the shield to releasably lock an axial position of the shield with respect to the elongated hollow barrel, wherein at a first locking position the shield covers the entirety of the hypodermic needle, at a second locking position the shield partially covers the hypodermic needle, and at a third locking position the shield is fully retracted so as to fully expose the hypodermic needle.

7. The syringe with an adjustable needle shield as recited in claim 6, wherein the hollow cylindrical mount has an axial length less than an axial length of the shield.

8. (canceled)

9. The syringe with an adjustable needle shield as recited in claim 6, wherein the hollow cylindrical mount and the shield are concentric with respect to one another.

10. The syringe with an adjustable needle shield as recited in claim 9, wherein the hypodermic needle extends along an axial center of each of the hollow cylindrical mount and the shield.
